(19) United States
(12) Patent Application Publication
(10) Pub. No.: US 2023/0117131 A1
(43) Pub. Date: Apr. 20, 2023

(54) Optimization of Radionuclides for Treatment of Cutaneous Lesions

(71) Applicants: Kenneth Wesley Brooks, Roswell, GA (US); Zachary Gray Carter, Woodstock, GA (US); E. J. Asbury, III, Marietta, GA (US)

(72) Inventors: Kenneth Wesley Brooks, Roswell, GA (US); Zachary Gray Carter, Woodstock, GA (US); E. J. Asbury, III, Marietta, GA (US)

(21) Appl. No.: 17/967,806

(22) Filed: Oct. 17, 2022

Related U.S. Application Data
(63) Continuation-in-part of application No. 17/503,350, filed on Oct. 17, 2021.
(60) Provisional application No. 63/405,890, filed on Sep. 13, 2022.

(51) Int. Cl. A61N 5/10

(52) U.S. Cl. CPC A61N 5/1029; A61N 2005/1094

(57) ABSTRACT

The present invention provides radioactive dermatological patch designed to topically treat cutaneous skin lesions in patient tissue. The radioactive dermatological patch includes a layer of a radionuclide with a nonreactive binding agent to form a treatment wafer. The treatment wafer is then placed within the radioactive dermatological patch. The radioactive dermatological patch includes a high Z distal shielding layer placed adjacent the side of treatment wafer away from the patient tissue. The distal shielding layer attenuating the energy of the radionuclide from the environment external to the patient. The patch further includes a high Z proximal patient shielding layer placed between the patient tissue and treatment wafer.

### REFERENCES CITED

### OTHER PUBLICATIONS

1. https://www.world-nuclear.org/information-library/non-power-nuclearapplications/radioisotopes-research/radioisotopes-in-medicine.aspx, Accessed October 2021.
2. https://www.aad.org/media/stats-skin-cancer, Accessed October 2021.
3. J.D. Lee et al, Radionuclide Therapy of Skin Cancers and Bowen’s Disease Using a Specially Designed Skin Patch, The Journal of Nuclear Medicine, Vol. 38 No. 5. May 1997.
4. K.B. Park et al, U.S. Pat. number 5,871,708, Feb. 16, 1999. Definitive and Postoperative Radiation Therapy for Basal and Squamous Cell Cancers of the Skin: Executive Summary of the American Society for Radiation Oncology, Clinical Proactive Guideline.
5. Likhacheva, Awan, M., Barker, C. A., Bhatnagar, A., Bradfield, L., Brady, M. S., Buzurovic, I., Geiger, J. L., Parvathaneni, U., Zaky, S., & Devlin, P. M. (2020). Definitive and Postoperative Radiation Therapy for Basal and Squamous Cell Cancers of the Skin: Executive Summary of an American Society for Radiation Oncology Clinical Practice Guideline. Practical Radiation Oncology., 10(1), 8-20. https://doi.org/10.1016/j.prro.2019.10.014
6. John Y.S. Kim, et al, Guidelines of care for the management of basal cell carcinoma, JAAD, Volume 78, Issue 3, p540-559, Mar. 1, 2018.
7. John Y.S. Kim, et al, Guidelines of care for the management of cutaneous squamous cell carcinoma, JAAD, Volume 78, Issue 3, p540-559, Mar. 1, 2018.
8. U.S. Pat., 5,871,708 Issued Feb. 16, 1999 to Park, et. al.
9. https://www.energy.gov/articles/department-energy-provide-16-million-isotopeproduction-rd.
10. https://www.novartis.com/news/media-releases/novartis-completes-tender-offeradvanced-accelerator-applications-sa-and-announces-commencement-subsequentoffering-period.
11. M.J. Salgueiro, H. Duran, M. Palmieri, R. Pirchio, J. Nicolini, R. Ughetti, M.L. Papparella, G. Casale, M. Zubillaga, Design and bioevaluation of a 32P-patch for brachytherapy of skin diseases, Applied Radiation and Isotopes, Volume 66, Issue 3, 2008,Pages 303-309, ISSN 0969-8043,
12. Sudhir Kumar, P. Srinivasan, S.D. Sharma, Sanjay Kumar Saxena, A.K. Bakshi, Ashutosh Dash, D.A.R. Babu, D.N. Sharma, Determination of surface dose rate of indigenous 32P patch brachytherapy source by experimental and Monte Carlo methods, Applied Radiation and Isotopes, Volume 103, 2015, Pages 120-127, ISSN 0969-8043,
13. DALE. (1985). The application of the linear-quadratic dose-effect equation to fractionated and protracted radiotherapy. British Journal of Radiology, 58(690), 515-528. https://doi.org/10.1259/0007-1285-58-690-515
14. U.S. NUCLEAR REGULATORY COMMISSION REGULATORY GUIDE 8.39 REVISION 1
15. Debois JM. Cesium-137 brachytherapy for epithelioma of the skin of the nose: experience with 370 patients. J Beige Radiol. 1994 Jan;77(1):1-4. PMID: 8005995.
16. Pashazadeh, Landes, R., Boese, A., Kreissl, M. C., Klopfleisch, M., & Friebe, M. (2020). Superficial skin cancer therapy with Y-90 microspheres: A feasibility study on patch preparation. Skin Research and Technology, 26(1), 25-29. https://doi.org/10.1111/srt.12758
17. Gamma Ray Dose Constants http://www.iem-inc.com/information/tools/gamma-ray-dose-constants
18. Dale, & Jones, B. (1998). The clinical radiobiology of brachytherapy. British Journal of Radiology, 71(MAY), 465-483. https://doi.org/10.1259/bjr.71.845.9691890

### CROSS-REFERENCE TO RELATED APPLICATION

This application claims the benefit of U.S. Provisional Application Serial No. 63/405,890 filed on Sep. 13, 2022, and is a continuation in part of U.S. Utility Application Serial No. 17/503,350 filed on Oct. 17, 2021, the entirety of each is incorporated herein by reference.

### BACKGROUND OF THE INVENTION

### 1. Field of the Invention

The present invention relates to the technical field of radiation therapy and specifically within this field to radionuclide therapy in the form of a superficial apparatus or a patch used to externally deliver radiation dose in the form of energetic ionizing radiation particles emanating from radioactive elements and the optimal configuration thereof. In particular, the present invention relates to a small superficial apparatus which contains the optimal mixture of radioisotopes and is attached to the surface of the skin or other body part to treat lesions in each range with a given mixture of isotopes.

### 1.1 Radiotherapy and Radiobiology

### 1.1 The Therapeutic Window

In the field of radiation therapy, radiation in many forms is directed towards lesions both benign and malignant which are destroyed or intended to be destroyed by the dose of radiation prescribed by a clinician. One of the fundamental discoveries of the field of “Radiobiology” or radiation biology in the 20^{th} century was that energetic radiation particles are more efficient at killing tumor cells and the cells of other malignant lesions in the mammalian body than they are at killing normal or healthy cells. This is due to the fact that healthy cells have a greater ability to self-repair than do the tumor cells, and hence are able to repair DNA strand breaks from the radiation particles while the tumor cells are not as effective in such self-repair. This fact of nature leads to the concept of a “therapeutic window” also known as the “therapeutic ratio,” which refers to the ratio of the number of malignant cells killed to the number of healthy cells killed by a given radiation dose applied equally to both specimens. Further sparing of healthy tissue can be affected by optimizing the geometric arrangement and the types and energies of radiation sources applied to the tumor relative to the healthy cells. It is the role of the radiation oncologists, radiation physicists and engineers at device vendors to consider how to best arrange a given set of radiation properties of a radiation source in such a way as to produce a maximal tumor destruction and a minimal normal adjacent tissue destruction. Each new technology is accepted or not based upon the ability of the technique to expand this window for a given condition by creating more destruction within lesions and less damage to normal tissues. The therapeutic window for cutaneous lesions is well established for traditional delivery mechanisms comprised of large expensive capital devices mostly purchased by large hospitals and medical centers. The point of care where the skin lesion is diagnosed is almost always at a dermatologist office where these large pieces of equipment are typically not present. Certain cohorts of patients are not good surgical candidates, and for these a reliable source of treatment which can maintain the therapeutic window of large radiation devices, but which is logistically condensed down is a novel idea and would be a welcome alternative to surgery or weeks of visits to a radiation bunker of a large hospital.

### 1.2 Brachytherapy

“Brachytherapy” is a term well known to those skilled in the art of radiation therapy which refers to the use of radioisotopes to treat tumors and other malignant lesions in the patient’s body. Brachytherapy may be classified into the general categories of “Low Dose Rate” (LDR) and “High Dose Rate” (HDR), which have different radiobiological characteristics owing to the different timescales over which they deliver the radiation dose. Superficial lesions have typically been treated in the past with either high dose rate brachytherapy sources such as Iridium-192 or other high dose rate X-ray tubes and linear accelerators. Some examples of the use of low dose rate brachytherapy for skin cancer are provided in Section 2.

### 1.2.1 Biologically Effective Dose

Over the past few decades, it has become common in radiotherapy literature to rely on the concept known as the “Biologically Effective Dose” or (“BED”) which is an extremely significant factor for comparing local control of tumors and other malignant lesions using different delivery technologies and platforms. The BED is a number which comes from a function whose variables include the quantity of radiation dose imparted in the tissue as well as the timeframe over which such radiation dose is imparted to the patient. For example, a single dose of 10 Gray (“Gy”) imparted to a tissue will have a different BED if it is delivered within 2-3 minutes as opposed to the same dose of 10 Gy being delivered continuously over 2-3 days, despite the fact that the total (or absorbed) dose is exactly the same. Hence, in order to compare the effectiveness of low dose rate brachytherapy for skin cancer to other established skin cancer treatments such as X-ray machines, linear accelerators, and high dose rate brachytherapy sources, one must compare the BED of each of these techniques.

### 1.3 “Depth Dose” and the Quality of Radiation Beams

A further important concept in radiotherapy is the concept of the “depth-dose” distribution, also known as the “percent depth dose” or (“PDD”). The PDD describes how deeply a given radiation field penetrates into the target tissue. A “high PDD” or “hard beam” refers to a radiation field which penetrates deeply into the target material and deposits significant dose at deep depths. A “low PDD” or “soft beam” refers to a radiation field that deposits the majority of its energy at shallow depths and rapidly declines in strength or fluence at deeper depths in the material. This concept may be applied to the deposition of radiation dose in tissue from external beam treatments or from the radiation dose imparted by radioisotopes. The percent depth dose is also sometimes referred to as the “beam quality,” meaning that the quality of the radiation for therapeutic purposes is directly related to its ability to impart radiation dose in deep areas of the target tissue(s). For the treatment of superficial lesions, a radiation beam which can penetrate to approximately 1 centimeter (cm) depth from the tissue surface and impart significant radiation energy at that depth is considered to be sufficient to cover the microscopic disease from most superficial lesions. This is because the epidermis and dermis exist to this level and most of the cutaneous lesions present somewhere from the surface of the skin with shallow extent, typically associated with basal cell carcinoma and then deeper down to 1 cm and sometime beyond for squamous cell carcinoma. Hence, in most cases the treatment of superficial lesions is carried out with large, expensive machines such as therapeutic X-ray tubes and megavoltage (MV) electrons from linear accelerators which are capable of sending significant radiation dose to this depth in tissue.

### 1.4 Radiation Protection and the Nuclear Regulatory Commission

The United States Nuclear Regulatory Commission (“NRC”) governs the use of reactor byproduct and other manmade radionuclides in the healing arts under section 10 C.F.R. 35 of the United States code. The publication entitled “NUREG 8.39” from the NRC gives specific instructions on determining the “release criteria” for patients who have been administered radionuclides for medical purposes. This release criterion for a given radionuclide refers to a level of radioactivity in the patient of said radionuclide that is designated by the NRC to be “safe” if the patient is released into the general public with specific safety-related instructions. Equation 3 of NUREG 8.39 may be used to calculate the release criteria for a patient administered a medical radiation device containing a short-lived radioisotope. The allowable radioactivity in the device is any activity that results in a member of the public receiving an estimated total dose of 5 mSv (milliSieverts) or less attributed to the patient with the device. This release criteria may be achieved by one of two means; the first means is to build shielding into the device around the radioactive material such that the exiting flux of radiation from the source housing is attenuated by the shielding in the housing. The second method is to simply reduce the activity level of the source itself until the release criteria is met. Any combination of these two methods may be used to achieve the designated release criteria calculated with Equation 3 of NUREG 8.39.

The U.S. NRC provides a computer code called VARSKIN which may be used to calculate skin doses from the presence of radionuclides in close contact to the skin. This code has been well validated in the radiation protection community and is presently considered as a “gold standard” for the calculation of superficial radiation dose from radionuclides. Although not written explicitly for therapeutic dose calculation purposes, the VARSKIN code may also be used to calculate the PDD curves that are created by radionuclide(s) near the skin surface [16].

### 1.5 Production of Radionuclides

Radionuclides have been used in medicine from early in the history of their discovery at the turn of the 20th century. Radionuclides as referred to here are either excited byproducts from nuclear fission whose chemical properties place them within the lanthanides in the periodic table of the elements or are specially created targets placed in a high neutron or photon flux of a nuclear reactor or a high energy charged particle accelerator. The irradiation of the targets allows the selected elements within the target to transmute from the energy imparted by the incident particles ultimately into a medically useful isotope. Regardless of how the resultant radionuclides are created, they are unstable and decay by either alpha particle, beta particle or photons or some combination of all three and can create a chain of decay particles and daughter products. In some cases, special isotopes decay with particles and energies which are suitable for use with many diseases in the human body. [1]

Originally produced from nuclear reactor fission products isolated chemically and prepared for medical use, the production of radionuclides was largely confined to nuclear reactors with specialized processing. However with the advancement of particle accelerators becoming more compact and more cost effective than a nuclear reactor, radiopharmaceuticals for diagnostic and therapeutic uses have become reliable as a safe and steady supply of certain workhorse isotopes such as Technetium-99 for cardiac imaging, Iodine-131 bound into a monoclonal antibody drug used for Thyroid treatment and Flourine-18 for producing Fluorodeoxyglucose (FDG) for Positron Emission Tomography (PET) scanning for cancer assessment as just a few of the high volume examples coming into mainstream worldwide over the last several decades. [1]

More uses for radionuclides have arisen from recent innovations in this area introducing a growing list of nuclides enabled by the ability to make these novel isotopes having optimized energy decay properties for certain diseases and delivery technologies produced more conveniently than within a nuclear reactor. As the production and biochemical delivery mechanisms have evolved along with all medical science and radiochemistry over the last 30 years more factors are coming together to enable a more selective and strategic use of isotopes to be integrated into sophisticated macro molecules or otherwise chemically bound to elements which have physiological importance for applying the specific decay scheme energy from the isotope for a given medical condition.

In the United States, the U.S. Department of Energy has been actively supporting the development of advanced accelerators capable of replacing nuclear reactors for these strategically crucial isotopes relied upon to save lives in medicine currently. The result of these concentrated development efforts has companies and established laboratories expanding to develop new technology to address these priorities. One such effort started from the CERN laboratory led to the development of Leuticium-177 in a public company called Advanced Accelerator Applications which was purchased by the Swiss drug company Novartis° for $3.5 billion in about 2019. Since that time a flurry of activity has occurred, and the technical development of special isotope production has risen sharply attempting to meet the demand for these ‘boutique’ isotopes for a given medical condition and/or drug or agent which can deliver it to a lesion.

### 1.6 The Inability of the Present Scarce Radiotherapy Resources to Meet the Needs of Patients with Superficial Lesions

The motivation for using an isotope instead of a large linear accelerator or even x-ray device designed for therapeutic use has to do with the high prevalence of skin cancer in the developed world, which for reference is cited in literature at approximately 3.5 million patients having over 5.5 million lesions treated annually in the United States alone in 2019 and growing. This compares to around 1.2 million patients for all other solid tumors in the body annually in the United States or somewhere on the order of 5 times as many skin lesions to treat as all other solid tumors in the body in the Unites States. [2] The prevalence of skin cancer or cutaneous skin lesions extrapolates into a very large number of patients on a global scale impacted by this disease annually.

The standard of care involves dermatologists using surgical techniques such a MOHS microsurgery, conventional excision, curettage, destruction, and radiation therapy using megavoltage electrons from linear accelerators at 4 or 6 MeV and x-rays ranging in energy from 50 to 100 kVp. In addition to their being a very large number of lesions to treat annually, using isotopes can allow all sizes of dermatology practices to consider their use on patients which may not be suitable for surgical treatment due to contraindications such as blood thinners and who also may not be able to travel to a clinic from 15 to 30 times for treatments from a linear accelerator or an X-ray system. In the US, large practice groups can consolidate high volumes of patients from their satellite smaller office which are suitable for radiation therapy to help offset the overhead and cost of creating a dedicated skin cancer treatment facility within their practice at each location. Use of a device that could replace those large machines can effectively democratize the use of radiation therapy, particularly radionuclide therapy, and spare unnecessary or unwanted surgeries all at a smaller logistical impact to the dermatology practices. The necessary technologists, physicists, and licensed isotope-prescribing physicians are readily available to properly support such activities in dermatologist’s offices at the point of care and diagnosis to avoid patients needing to navigate a large hospital nuclear medicine department or a radiation oncology department. This technology can also enable a truly multidisciplinary approach to provide more optimal care for appropriate portions of the skin cancer patient population.

### 2. DESCRIPTION OF THE RELATED ART

Application of radioisotopes in a patch for cutaneous lesions was already conceived using a lower energy isotope Holmium-166. Holmium’s decay scheme while reported to be useful in one publication, demonstrated in that publication that the electrons emitted are not penetrating enough to closely replicate a megavoltage electron beam from a linear accelerator for such lesions or cover the same depth as keV energy x-rays [3, 4]. In fact, the published results from the authors reported negative cosmetic sequalae associated with the use of a Ho-166 patch which are consistent with the low energy beta particle depositing their energy within the first 2-3 mm of cutaneous tissue and all within periods of 1 -2 hours. The use of Ho-166 for skin lesions was apparently stopped and is still not in widespread use today. This result is apparently partially due to the suboptimal energy of the electrons emitted for use in cutaneous lesions from Ho-166. Very effective surgical techniques and external beam radiation sources give effective control without causing such significant side effects as discussed in the Ho-166 study.

A wearable radiation patch design has also been published using the isotope Phosphorous-32 (“P-32”) [11][12]. In this example of prior art, a 1-millimeter thick radioactive laminated paper patch was produced. The patch did not have built-in shielding except for the small amount of plastic in the laminating material. The beta emissions from the P-32 source are deemed to be not penetrating enough to be a viable solution for the treatment of most superficial lesions which need to be irradiated down to a depth of 7-10 mm, and hence the commercial development of the P-32 patch did not succeed.

Another superficial patch therapy concept was described in the publication by Pashazadeh et. Al. 2019. Paper was again used as the substrate upon which the radioactive material was embedded. The source was selected as Yttrium-90 (“Y-90”) which is a high energy beta particle (electron) emitter. This paper was one of the first publications to use Y-90 for cutaneous lesions externally in a topical delivery mechanism.

Non-melanoma skin cancers for example typically do not exhibit excessive vascularity at least in their earlier stages thus radioembolization techniques would not be applicable. These lesions are very well controlled with good cosmetic results using a radiation beam of 4-6 MeV electrons and 50-75 kVp x-rays as has been described in many publications and in recent guidance by the American Society of Radiation Oncology (“ASRO”) [5] and the American Academy of Dermatology (“AAD”). Also, both 4-6 MeV electrons and kilovoltage x-rays have been included in the United States (National Comprehensive Cancer Network) and the equivalent European clinical guidelines for cancer treatment for cutaneous lesions for many decades.

In one prior art publication, low dose-rate brachytherapy was used to treat epithelioma of the nose using Cesium-137, a classical isotope that has been used to treat cancer since the early 1900s [15]. While this treatment achieved excellent results in the local control of the cancers, the isotope Cs-137 that was used has many problems associated with its long half-life.

Accordingly, what is needed in the art is an efficient means to take advantage of the range of radionuclide isotopes available for treatment of cutaneous skin lesions. Cutaneous skin lesions in the tissues of human beings and other mammals may take the form of malignancies such as melanoma or non-melanoma basal cell and squamous cell carcinomas, lymphomas, and several other skin lesions which require treatment. The apparatus should provide a ready means to deliver ionizing particles emanating from radionuclide isotopes, which may be newly discovered or well-established isotopes suitable for use with many diseases in the human body and for veterinary use in other mammals. Also needed is a methodology for the selection of the Radionuclide isotope or combinations of Radionuclide isotopes for use within the apparatus for patient treatment of a given cutaneous lesion.

### SUMMARY OF THE INVENTION

The disadvantages of the prior art are overcome by the present invention which, in one aspect is a radioactive dermatological patch, the patch designed to topically treat cutaneous skin lesions in patient tissue. The patch including a layer of a radionuclide with a nonreactive binding agent to form a treatment wafer. The treatment wafer placed within the radioactive dermatological patch. A high Z distal shielding layer placed adjacent the side of treatment wafer away from the patient tissue. The distal shielding layer attenuating the energy of the radionuclide from the environment external to the patient. And the patch further including a high Z proximal patient shielding layer placed between the patient tissue and treatment wafer.

In another aspect of the invention, the radioactive dermatological patch includes an additional window shielding layer of high Z shielding placed between the treatment wafer and the proximal patient shielding layer. The window layer of high Z shielding including a cutout opening within the window shielding layer in the shape of the cancerous tissue to be treated by the radionuclide and the window layer including a substantially solid layer of high Z shielding above the remaining healthy patient tissue.

In another aspect of the invention, the radioactive dermatological patch includes an additional window shielding layer of high Z shielding placed between the distal shielding layer and the proximal patient shielding layer. The window shielding layer including a cutout opening within the window shielding layer in the shape of the cancerous tissue to be treated by the radionuclide and the window layer including a substantially solid layer of high Z shielding above the remaining healthy patient tissue. And werein the treatment wafer is applied withing the cutout of the window shielding layer opening.

In another aspect the radioactive dermatological patch includes the radionuclide of at least one of: Na-24, Ga-66, or any combination thereof.

In another aspect the radioactive dermatological patch wherein the nonreactive binding agent is comprised of a silicone rubber.

In another aspect the radioactive dermatological patch wherein the window shielding layers of high Z shielding is comprised of at least one of: lead, tungsten, iron, silver, gold, platinum, copper, brass, or any combination thereof.

In another aspect the radioactive dermatological patch wherein the window shielding layers of high Z shielding is comprised of at least one of: lead, tungsten, iron, silver, gold, platinum, copper, brass, or any combination thereof.

In another aspect the radioactive dermatological patch wherein the radionuclide and nonreactive binding agent are formed in a substantially circular treatment wafer within a mold.

In another aspect the radioactive dermatological patch wherein the radionuclide and nonreactive binding agent are formed in a substantially elliptical wafer within a mold.

In another aspect the radioactive dermatological patch of wherein the radionuclide and nonreactive binding agent are formed in a custom shape corresponding to the shape of the cutaneous skin lesions in the patient tissue.

In another aspect the radioactive dermatological patch wherein the treatment wafer shape corresponds to a portion of the upper cranium of the patient.

In another aspect the radioactive dermatological patch wherein the distal shielding layer is comprised of at least one of: lead, tungsten, iron, silver, gold, platinum, copper, brass, or any combination thereof.

In another aspect the radioactive dermatological patch wherein the proximal shielding layer is comprised of at least one of: lead, tungsten, iron, silver, gold, platinum, copper, brass, or any combination thereof.

In another aspect the radioactive dermatological patch wherein an adhesive binds at least one of: the proximal shielding layer to the window shielding layer, the distal shielding layer to the window shielding layer, the proximal shielding layer to the treatment wafer, the distal shielding layer to the treatment wafer, the proximal shielding layer to the distal shielding layer.

In another aspect the radioactive dermatological patch wherein an adhesive binds at least one of: the proximal shielding layer to the window shielding layer, the distal shielding layer to the window shielding layer, the proximal shielding layer to the distal shielding layer.

In another aspect the radioactive dermatological patch wherein the proximal shielding layer substantially attenuates the electron energy emitted from the radionuclide.

These and other aspects of the invention will become apparent from the following description of the preferred embodiments taken in conjunction with the following drawings. As would be obvious to one skilled in the art, many variations and modifications of the invention may be affected without departing from the spirit and scope of the novel concepts of the disclosure.

### BRIEF DESCRIPTION OF THE DRAWINGS

FIG. 1 is a perspective view of a cutaneous lesion as shown on a human forearm.

FIG. 2 is a perspective view of a cutaneous lesion as shown on a human forehead.

FIG. 3A depicts a table of Electron and Photon Emitting Radionuclides compared to 6 MeV electrons and 100 kVp X-ray sources for treating cutaneous lesions.

FIG. 3B depicts a graphical depth dose comparison of commonly used Xray & Gamma Ray sources.

FIGS. 4A, 4B, and 4C compare Ho-166 to Y-90 and Lu-166 in terms of decay spectra of electrons.

FIG. 5A depicts a radionuclide patch design which allows dosimetry optimization.

FIG. 5B depicts a radionuclide patch design which allows dosimetry optimization to be custom shaped for a given body site.

FIGS. 6A - 6B depict a radionuclide patch design which allows dosimetry optimization via a flexible patch design shaped for large areas as well as contour changes.

FIG. 7 depicts the percent depth dose curves for the radionuclides Na-24 and Ga-66 vs the percent depth dose curves of the accepted external beam skin treatment techniques.

FIG. 8 depicts the percent depth dose curves for Na-24 unshielded and for Na-24 with 0.3 cm H20 shielded and 0.6 H20 shielded.

FIG. 9 depicts the percent depth dose curves for Ga-66 unshielded and for Ga-66 with 0.3 cm H20 shielded and 0.6 H20 shielded.

FIG. 10 depicts the beta PDD curves from a selection of isotopes computed in VARSKIN compared to the accepted external beam skin treatment techniques.

FIG. 11 depicts the gamma photon PDD curves from a selection of isotopes computed in VARSKIN compared to the accepted external beam skin treatment techniques.

FIG. 12 depicts the “Total” PDD curves, in other words the PDD curves resulting from the summation of the dose from beta particles and the dose from gamma particles at each depth, from a selection of isotopes computed in VARSKIN compared to the accepted external beam skin treatment techniques.

### DETAILED DESCRIPTION OF THE INVENTION

The present invention provides an efficient means to take advantage of the range of radionuclide isotopes, referred to herein as radioisotopes or radionuclides, available for treatment of cutaneous skin lesions of the human body. The present invention relates to the technical field of radiation therapy and specifically within this field to radionuclide therapy in the form of a superficial apparatus or a patch used to externally deliver radiation dose in the form of energetic ionizing radiation particles emanating from radioactive elements and the optimal configuration thereof. In particular, the present invention relates to a small superficial apparatus which contains the optimal mixture of radioisotopes and is attached to the surface of the skin or other body part to treat lesions in each range with a given mixture of isotopes. The present invention proffers a solution to the therapeutic depths that radioisotopes can achieve by optimizing both the selection of the proper radioisotope for superficial brachytherapy treatment as well as the geometrical arrangement of the source and its housing. The present invention also relates to the process of using the information about the lesion and novel isotopes to 1) selectively design a mixture of isotopes whose radiation decay processes can provide full dose coverage of the lesion, and 2) based upon the mixture of the isotopes, also choose a thickness of material comprising the interface between the lesion and the radiation source to give a radiological bolus effect and further 3) to design the back side or externally facing portions of the patch to contain proper shielding for the timeframe and anticipated exposure risks of the patient or any proximal persons in contact with the patient during treatment.

The key design objective of a wearable patch of radionuclides is the ability of the isotope to control the radiation with a favorable side effect profile relative to other radiation and surgical alternatives. With the advent of new isotopes, the field is advancing, and it is a chance to reconsider this approach for the application to cutaneous lesions. The wearable patch of this invention allows for a delivery mechanism for the radiation to be conformed to the shape of the lesion in the superficial plane and then optimized in depth by considering the unique properties of various available isotopes and the clinical condition of a given patient’s lesion. The invention also covers optimizing the depth-dose curve with a built-in bolus feature on the side proximal to the patient which serves to remove undesirable particles from the raw, unfiltered radiation source while allowing desirable particles to pass through and deliver the therapeutic radiation dose, and a covering on the side distal to the patient that also serves as a safety shield. The object of the invention is to provide this patch device capable of accepting various forms of radioactive substrates containing various radioisotopes and packaging them into the aforementioned device, the details of which will be described in the following paragraphs.

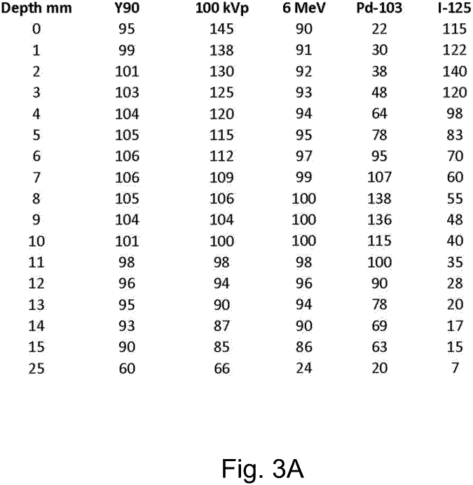

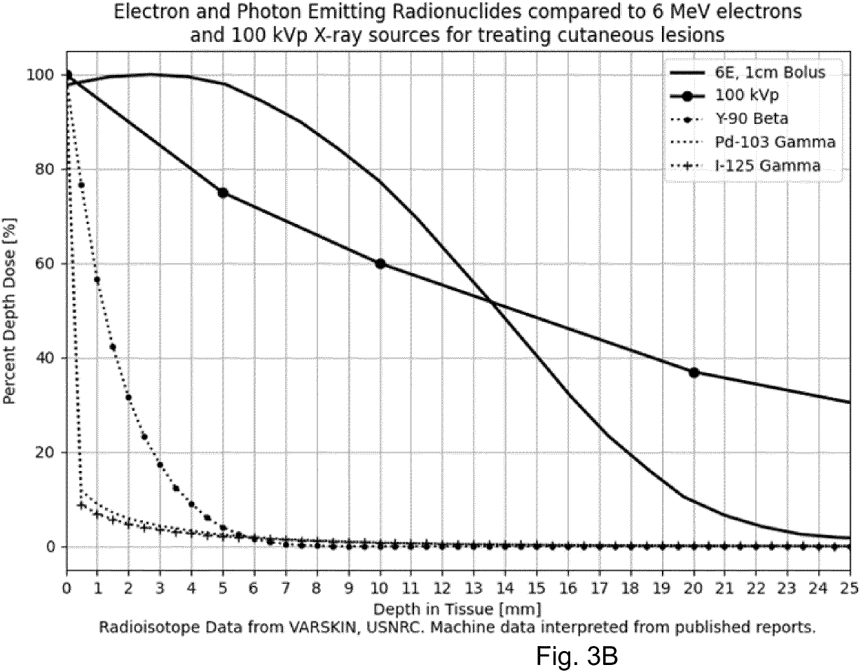

Our studies have shown that using a nuclide such as Yittrium-90, which has become widely established for radioembolization in recent years for cancerous and benign processes that involve excessive vascularity, one might more closely approach a depth dose curve of an electron 4-6 MeV linear accelerator beam or at least achieve something close to a keV x-ray source. FIG. 3A presents a table of Electron and Photon Emitting Radionuclides compared to 6 MeV electrons and 100 kVp X-ray sources for treating cutaneous lesions. FIG. 3B depicts a graphical depth dose comparison of commonly used Xray & Gamma Ray sources.

The disadvantages of the prior art are explained by the following statements: 1) the isotopes of P-32, Y-90, Ho-166, amongst others used in the prior art have percent depth dose (PDD) curves which are too shallow to cover the microscopic disease in most superficial lesions that present clinically. 1) The prior art did not take into account the effect of the radiation energy spectrum on the resultant percent depth-dose curve(s). 2) Many of the isotopes used in the prior art were too long-lived, or in other words their radiation half-lives are too long to be used conveniently in a superficial therapeutic radiation patch while ceasing to pose a danger to the public after the radiation treatment has concluded. 3) The previous radiation patches that have been published did not contain significant built-in self-shielding capabilities and hence may present a danger to the public for the duration of the patch treatment due to the unshielded or unattenuated flux of radiation emanating from the patch. 4) The prior art does not take into account the effects of BED, fractionation and source dwell time (or total treatment time per fraction) when compared to low dose rate brachytherapy with short-lived radiation sources. Each of these disadvantages of the prior art is discussed in more detail in the following paragraphs.

The isotopes of P-32 and Ho-166 are not suitable for a superficial therapy patch because their resultant percent depth-dose curves do not penetrate deeply enough into the tissue to cover the microscopic disease for most clinical skin cancer cases such as squamous cell carcinoma and basal cell carcinoma. The isotope Y-90 may be suitable for a therapeutic patch, but only for shallow lesions such as keloids as it has a fairly shallow PDD curve which reduces to 50% intensity within 2-3 mm. The isotopes Cs-137 and Co-60 may have nicely penetrating PDD curves due to their high gamma energy spectrum, but they are long-lived isotopes and hence are not good candidates for a disposable radiation therapy patch for that reason. Accordingly, the optimal isotope for treating lesions which reach up to 1 cm in depth will have a relatively short half life on the order of 10-24 hours while also having a high gamma or photon energy spectrum which results in a deep penetration of the percent depth dose curve in the patient’s tissue, and a low beta energy spectrum so that the emitted electrons do not negatively affect the PDD curve by depositing a large amount of dose within the first 1 mm and little dose at deeper depths. One example of such an optimal isotope would be Sodium-24.

A code developed by the U.S. Nuclear Regulatory Commission (“NRC”) called VARSKIN has been widely validated as accurate for human skin dosimetry. The use of VARSKIN to calculate a PDD from a skin patch has already been published for the isotope Yttrium-90 (Y-90) [16], and the same technique may be used for sodium-24 (“Na-24”) and Gallium-66 (“Ga-66”) as well as any other isotope contained in the VARSKIN database.

The problem with using longer-lived isotopes such as Cesium-137 and Iridium-192 to treat skin cancer is that the isotope continues to endanger the public long after the treatment of the cancer has concluded. Many of such long lived sources have a suitable depth dose characteristic, but are required to be packaged inside of a complex and expensive electromechanical delivery systems such as HDR afterloader machines or otherwise are required by NRC regulations to be located in a designated restricted “radiation area” while the treatment is underway and locked inside a vault or safe when not in use. Such sources can quickly expose staff to excessive amounts of radiation, for example by manual manipulation of Cesium sources and reusing them on different patients or during a malfunction of an HDR afterloader machine. Because they are longer lived, they also must be replaced as they eventually decay and become too weak creating another expensive and dangerous requirement to periodically replace these sources to maintain them at full strength.

The United States Nuclear Regulatory Commission (“NRC”) would not permit a patient to leave the licensee’s facility with a long-lived isotope such as Cs-137, so the patients would be required to spend long hours at the medical facility to complete their treatment. With short-lived radioisotopes, patients may be permitted by the NRC to go home with the isotope on their person since the isotope does not pose a great danger to the public and will decay to a harmless level of activity within a short time. In this manner, the medical treatment will begin at the licensee’s facility and the patient will continue to receive the benefit of the treatment at their home in the following days and weeks without causing a danger to the public, which is simply not possible nor allowed by NRC regulations for longer lived isotopes.

If a short-lived isotope such as Sodium-24 is used instead to treat the cancer, the isotope will decay to less than 0.1% of its original activity after 1 week has passed from the commencement of the treatment. Hence, in a similar manner to other isotopes such as I-131, Pd-103 and I-125 that have been given their own special “release criteria” in the NUREG 8.39 report, it is possible to calculate a “safe” activity of Sodium-24 that will result in a member of the public receiving less than 5 mSv of dose according to the requirements of 10 CFR 35. Hence, a “release criteria” which follows the recommendations of NUREG 8.39 may be defined for the Sodium-24 and other superficially applied isotopes which allows the patient to return to their home with the radiation patch on their person as long as they follow specific radiation safety instructions provided by the clinic. Short-lived isotopes may be shipped in very stable and safe radiation “pigs” or leaded, nearly impenetrable packages where the source may be stored until it is ready to be placed on the patient’s skin. Furthermore, short-lived isotopes with half-lives on the order of 10 -24 hours present a way of efficiently delivering BED units to the patient because a large percentage of the source will decay during the time frame of the treatment so most of the activity available in the source is being used for the treatment. Hence, these short-lived isotopes are preferable from both a regulatory and nonproliferation point of view as well as a radiobiological point of view for use in a superficial skin therapy patch.

An unshielded patch may be capable of providing an effective therapy, but the logistical processing and handling of the patch would present problems for the medical staff and transportation in addition to the fact that an unshielded patch may not be allowed to leave an NRC-licensed facility. The objective of a wearable patch should be to provide a deep enough dose deposition to cover the microscopic disease while also having an optimal half-life for the purposes of biological effectiveness and radiation protection as well as built-in shielding to meet the NRC release criteria and radiation safety guidelines. The prior art did not consider the need to have significant shielding built-in to the patch to protect the public from radiation during the radiation patch treatment. By including shielding on the externally facing portions of a medical radiation patch, the public will be protected and the activity of the radiation source may be adjusted while still maintaining a safe radiation level for the public if the shielding density and thickness is varied in reasonable proportion to the activity of the source.

Low dose rate brachytherapy is preferable to high dose rate brachytherapy and external beam radiation therapy because the malignant cells and the normal tissues have different rates of repair, and the normal tissues are more capable of repairing themselves from the low dose rate brachytherapy than the tumor cells are capable of self-repair. Furthermore, assuming an alpha/beta ratio of 10 Gy for tumor cells (which is commonly accepted to those skilled in the art of radiotherapy and may be demonstrated from the equations in [13]), it is possible to deliver a high BED with a relatively low total physical dose when one is using low-dose rate brachytherapy. A well-known example in radiotherapy is the widespread use of low dose rate brachytherapy to treat prostate cancer with permanently implanted radiation sources which deliver a very high BED in comparison to the given dose, effectively setting the standard for the highest therapeutic window achievable to date in prostate radiotherapy. In contrast, much more dose must be delivered to achieve the same BED when one is using high dose rate or fractionated external beam treatments. In other words, the low dose rate brachytherapy technique is more efficient at delivering BED units with less radiation dose units than are the other techniques.

In a first embodiment of the present invention, the binding agent into which the radionuclide is disbursed will be an initially liquid material and serve as a chemical solvent for the radionuclide and which can harden into a solid yet partially flexible material. The liquid binding agent and disbursed radionuclide may then be poured into a mold of a defined shape and depth to achieve a treatment wafer. The binding agent may be a liquid such as silicone into which the radionuclide is disbursed, and which will then harden after being poured into a mold of appropriate shape and depth. The treatment wafer comprising the binding agent and radionuclide can be molded with a circular shape of varying “standard” circular size diameters, for example 1 cm, 2 cm, 4 cm, etc. and an appropriate thickness. The treatment wafer may also be formed in a mold to achieve an elliptical shape with varying “standard” sizes and an appropriate thickness. The mold may also be designed with any custom shape to correspond to the shape of the skin carcinoma or lesion to be treated. The flexibility of the “hardened” silicone binding agent treatment wafer in conjunction with the malleable lead shielding layers allows the radionuclide patch to be readily shaped and conform to the patient skin contours at the treatment site. In other alternative embodiments, the binding agent may be hyaluronic acid or other hydrogel for which an activator may be added along with the isotope mixture which may then be appropriately solidified and shaped into a treatment wafer, or any combination of silicone and hydrogel. In yet another alternative embodiment of the present invention, the radionuclide may be deposited upon or embedded within a substrate to form a radionuclide with binding agent treatment wafer. The binding agent may be a cloth patch or tape as it known by those skilled in the art.

To form the radionuclide patch for application to the patient, the treatment wafer will then be placed between two sheets of “shield” material, such as lead (“Pb”). The thickness of the shield on the patient-facing side, the proximal shield, must be designed by physicist calculations that are isotope specific, as this is the side that needs to remove the beta particles from the beam while removing as few photons as possible. The distal shield on the external or outward facing side of the radionuclide patch will be significantly thicker (in terms of radiological thickness in grams per square cm, i.e. density times length) than the patient-facing shield. The distal (outward) shield is designed to protect members of the public from receiving excessive radiation dose from the radionuclide patch, while the patient’s body tissues will provide shielding on the proximal (inward) shield side. In other alternative embodiments, any shielding layer may be formed from an appropriate thickness of lead, tungsten, iron, silver, gold, platinum, copper, or brass, or any combination thereof.

The radionuclide patch is constructed of a binding agent material layer containing the radioactive isotope (“radionuclide”) and with a shielding layer between the radionuclide and patient, which is the proximal shield or inward shield, and with a shielding layer external to the patient between the radionuclide and outside environment, which is the distal or outward shield. The main points in the design of the patch are that both radionuclides Na-24 and Ga-66 must be considered a “sealed” source by the NRC (10 CFR 35). This means that the source must be sealed inside a container, the radionuclide or radioactive dermatological patch herein, that is designed not to rupture and potentially leak material into the environment.

In an alternative embodiment of the present invention, as tumors do not typically take on circular or elliptical shapes (although some do), the effect of the treatment wafer may be adjusted to match the actual shape of the tumor that is being treated. This may be done with an additional lead (Pb) sheet with an appropriate cutout, referred to herein as the window shield layer. The window shield layer is manually cut to shield normal tissues from the radiation while leaving a hole where the tumor is located. The window shield layer is then place between the treatment wafer and the proximate shield layer. The radionuclide patch is then formed by first an inner proximate shield layer, onto which a window shield layer is positioned, followed by the treatment wafer over the window shield layer cutout, and finally the outer distal shield layer.

The use to the window shield layer provides a readily high degree of customization to the shape of the desired treatment zone while utilizing the standard circular and elliptical shape treatment wafers produced in volume. In all embodiments herein, the malleable lead shielding layers with internal flexible treatment wafer allows the radionuclide patch to be readily shaped and conform to the patient skin contours at the treatment site. In another alternative embodiment of the present invention, the window shield layer is placed upon the proximate (inner) shielding layer. The radionuclide and binding agent are then poured or applied to the cutout within the window shield layer to an appropriate depth to form a custom shape treatment wafer. In this manner, the window layer placed upon the proximate shielding layer forms the mold for the now custom shaped treatment wafer. The steps in patch construction are reduced and the radioisotope material use is optimized.

### 1) Choice of Isotope(s)

In one embodiment, the present invention overcomes the problems associated with long-lived isotopes such as Cs-137 by using short-lived isotopes including but not limited to Sodium-24 (Na-24) and Gallium-66 (Ga-66), to provide low dose rate brachytherapy for skin cancer.

The radionuclide patch is a topical, non-invasive dermatological patch designed to treat cutaneous skin lesions with radiotherapy using a radioactive isotope. In principle, any radioactive isotope could be used in a patch, but the research presents two isotopes with the best dosimetry and practical characteristics: Sodium-24 and Gallium-66. One embodiment of this invention describes the novel use of Sodium-24 and Gallium-66 as the active ingredients in a medical radiation patch for superficial lesions.

Both Na-24 and Ga-66 emit beta (electrons) particles and gamma (photons) and both decay with very high energy above 1 MeV and even greater than 2 MeV for some of the photons in Na-24. In radiation therapy, the plot of the dose deposited into tissue (or a specified surrogate tissue medium like water) is tallied for the range of particles and energies of a given source of radiation at a range of clinically relevant depths. This tally is plotted as dose (or percent of maximum dose) versus depth for a given particle in a specific medium and is commonly called the Percent Depth Dose (“PDD”) curves. A PDD curve is a tool medical physicists and physicians use to describe how the intensity of radiation varies with depth in tissue, and hence how much radiation dose is absorbed by the tissue at different depths. The curve is the PDD in actual human skin and may be calculated via validated code models.

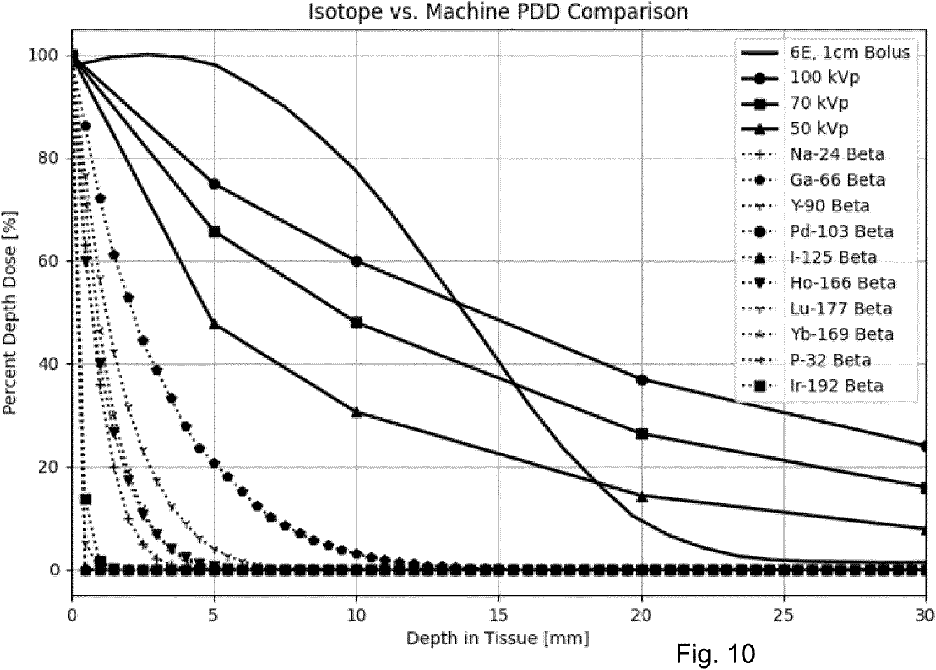

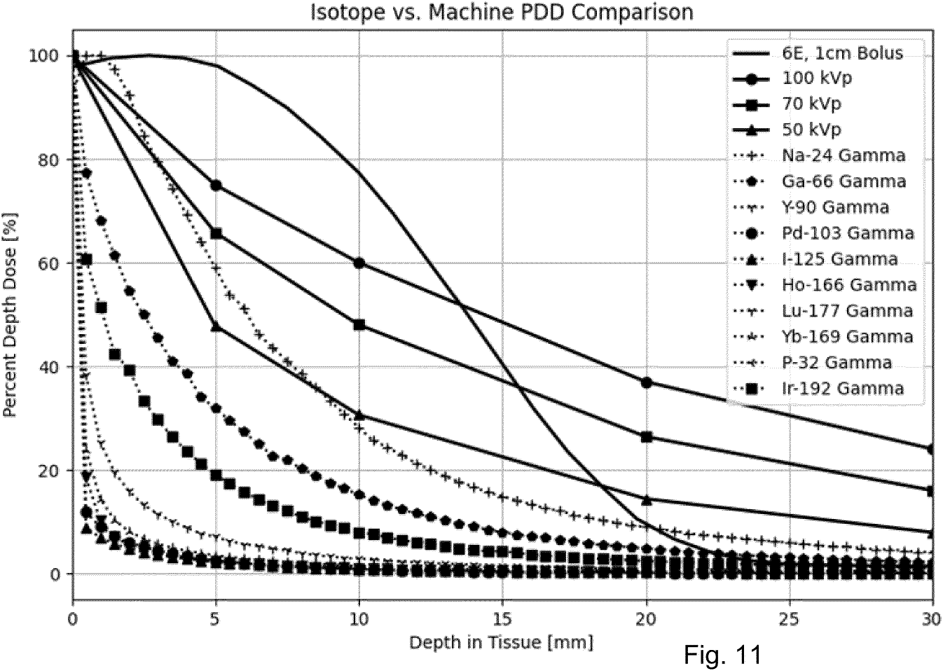

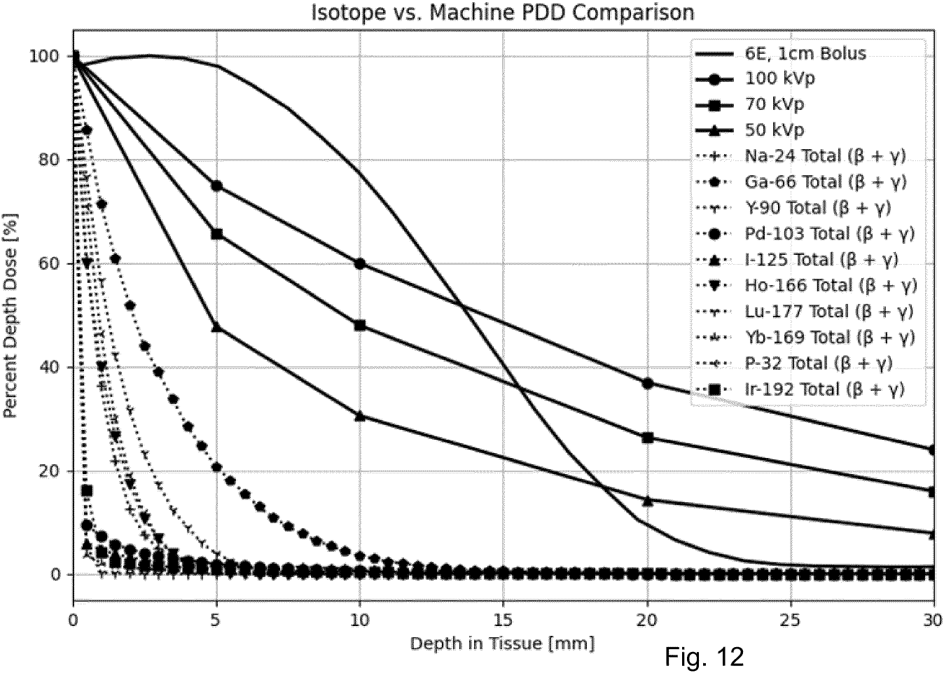

In the development of this invention, PDD curves were generated using the NRC’s VARSKIN code using nuclear decay data from the ICRP 107 publication. FIGS. 10, 11, and 12 show the calculated PDD curves for a selection of radioisotopes compared to the PDD curves from external beam treatment techniques. For the calculations, the isotopes are assumed to be distributed across a 1-millimeter-thick water-equivalent cylinder with a diameter of 2 centimeters, and no bolus is placed between the cylinder and the skin surface. FIG. 10 displays the PDD curves for the beta particle emissions from the radioisotopes. It is clear from the figure that Ga-66 (pentagon markers) provides the most penetrating beta PDD curve amongst the selected beta-emitting isotopes. FIG. 11 displays the gamma PDD curves for the same set of isotopes. Na-24 and Ga-66 provide the most penetrating gamma PDD curves. FIG. 12 displays the “total” PDD curves. The “total” PDD refers to the PDD curve that is generated by summing the dose from beta particles and the dose from gamma particles at each depth and calculating the PDD curve from the resultant sum. In this case, Ga-66 provides the most penetrating PDD curve.

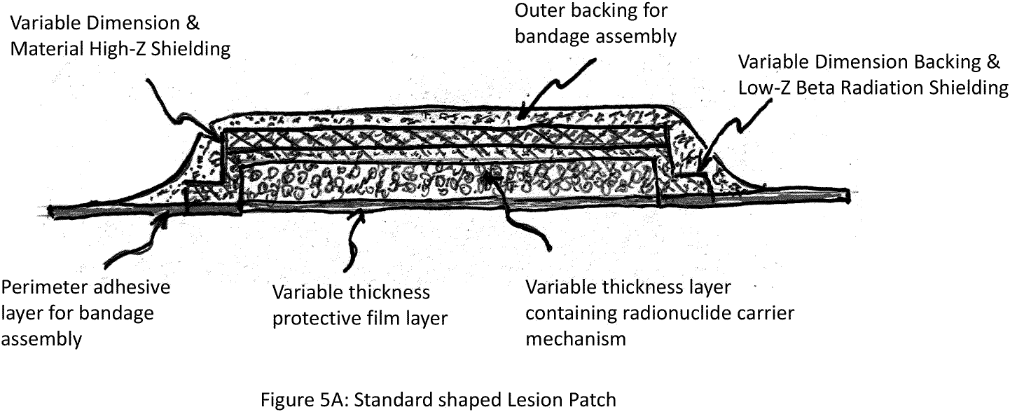

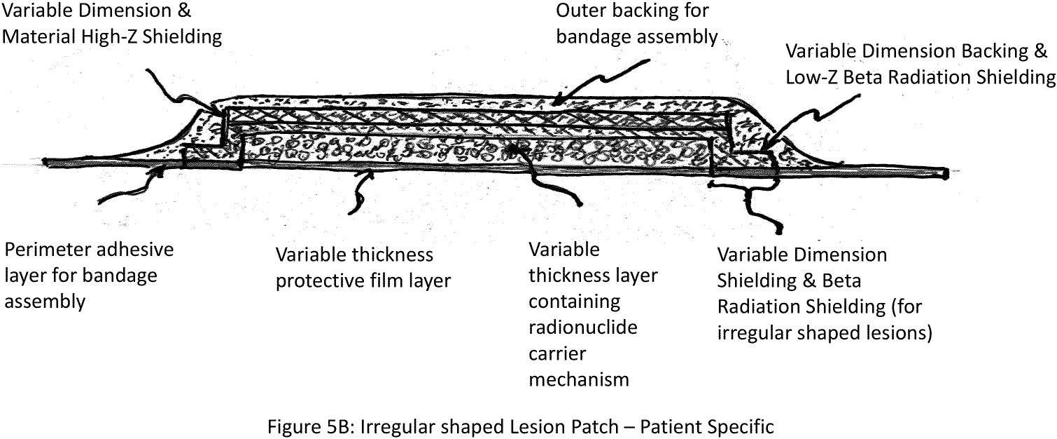

The invention discloses optimal use of radionuclides to fabricate a temporary covering or patch of materials containing sufficient radionuclide(s) also comprising a mixture of nuclides, with suitable decay schemes to mimic and closely resemble the dosimetry seen and used regularly with external beam electron and keV photon dosimetry. As depicted in FIGS. 5A and 5B, the cumulative, relative biological effectiveness (“RBE”) equivalent dose scheme of such an external beam radiotherapy device in cutaneous lesions is thus being achieved by a surface source of nuclides imbedded into the disclosed binding agent mechanism forming the active portion of a covering or patch that would be worn for a long enough time period to allow a curative prescribed dose to be deposited into the cutaneous lesions. Dose schemes including fractionation with a small number of sessions can be implemented and the corresponding changes to the nuclide patch loading and design considered for a series of treatments which would be tailored such that they are RBE equivalent to a 50 Gy total dose with 2 Gy per fraction in 25 fractions as is standard fractionation from an external beam source, for example. In an illustrative example using Sodium-24 and Gallium-66 in select mixture ratio with enough combined activity to provide an accelerated dose fractionation scheme that reduces the number of treatments from 30 down to 5, 3, or even a single treatment and covers the extent of the disease within the tissue depth.

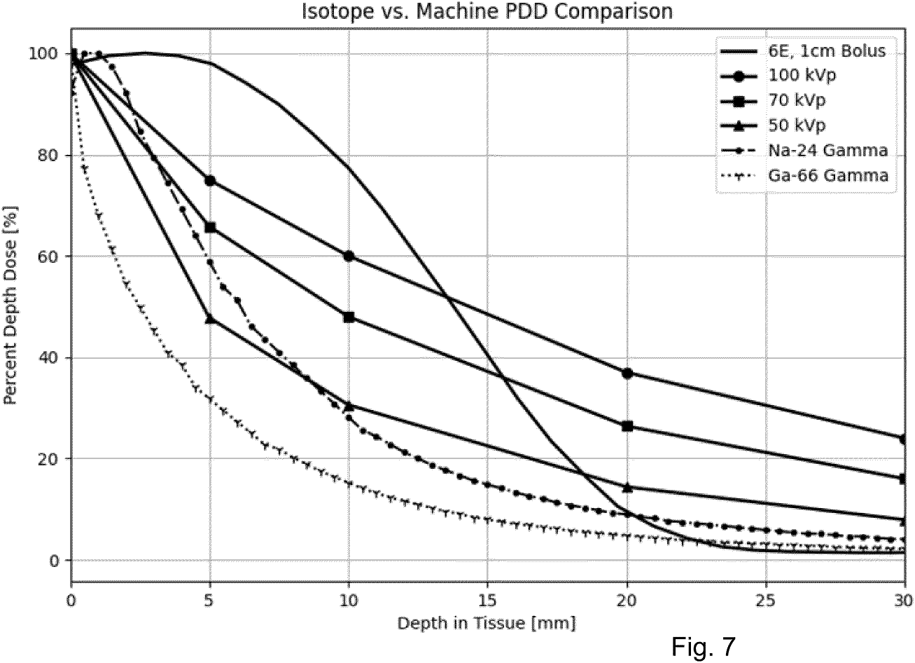

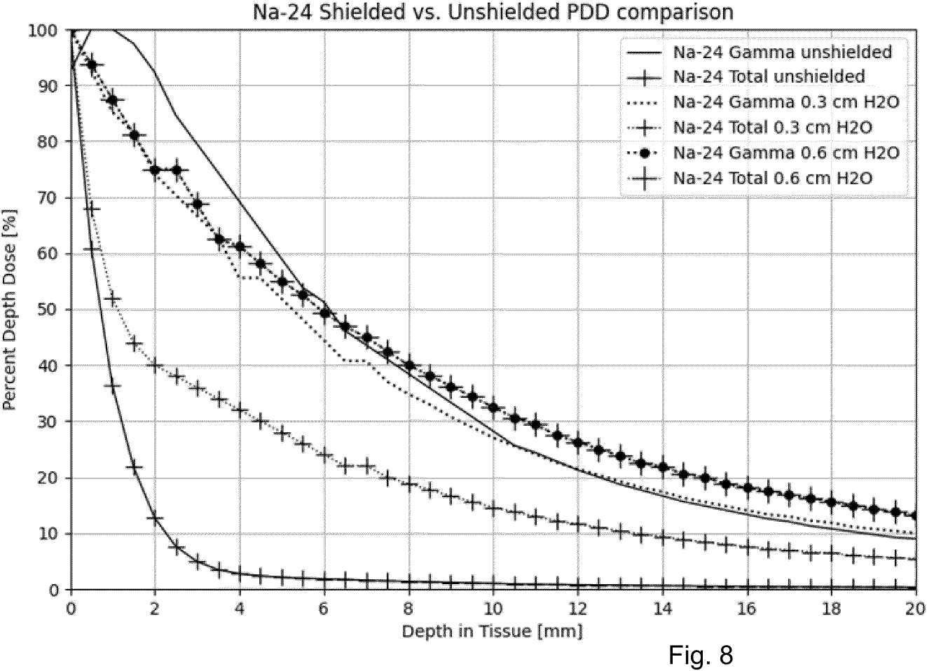

FIG. 7 presents the PDDs for the widely adopted non-surgical external beam radiotherapy skin treatment technology against the PDDs generated with VARSKIN for Na-24 and Ga-66 in human skin. For external beam techniques, 50 kVp is the most widely utilized technology with its associated PDD curve for human skin shown in FIGS. 7 & 8. For radiation oncology physicians, the “6 MeV + 1 cm bolus” curve would be the most commonly prescribed PDD curve, as this would be produced on an external beam linear accelerator which radiation oncologist have access to. For dermatologists, if radiation treatment is prescribed at all, the PDD curve will typically be from one of the “kVp” curves because these are produced by X-ray tubes which are much less expensive and mostly used in dermatology practices while the radiation oncologists typically use linear accelerators to deliver the prescribed dose. Based on the PDD curves presented in FIGS. 7 and 8, Ga-66 would be suited for shallower skin lesions while Na-24 would be the primary radionuclide for use in the dermatological patch for treatment of the majority of skin lesions which may extend to 1 cm in depth or so.

As shown in FIG. 7 and FIG. 8, the Ga-66 total curve is ideal for skin lesions of treating Keloid surgical beds and shallow basal cell carcinomas. A radionuclide patch properly designed with these two isotopes either alone or in combination could replace some of the currently used radiation machine based external beam sources dosimetrically and the convenience means its open to all sizes of dermatological practices.

Both the proximate and distal shield layers may be readily cut or formed from an appropriate thicknesses of lead sheet. The composite construction of the radionuclide patch allows the treatment facility to maintain a selection of lead sheet thicknesses. The appropriate thickness of lead sheet may then be selected for the distal, window, and proximate shield layers, and cut to appropriate dimensions. A treatment wafer of appropriate size may be obtained or constructed, and the radionuclide patch readily assembled and affixed to the treatment zone on the patient’s skin with commercially available adhesive cover bandages. In any embodiment herein, an adhesive may be used between the individual radionuclide patch layers to prevent relative shifting of the layers during patient treatment.

Sodium-24 (“Na-24”) is a radionuclide which has been used in the past for studies of peripheral blood flow, as well as being used in the oil and gas industry for pipe leak detection. Na-24 may be produced by irradiating “normal” Na-23 table salt (“NaCl”) with neutrons in a nuclear reactor or by irradiating sodium metaborate (“NaBO2”) with deuterium ions from a cyclotron. A cyclotron is a type of charged particle accelerator that can be used to produce medical isotopes. Deuterium is a hydrogen atom with an extra neutron. These are different production methods utilizing different reaction pathways to arrive at Na-24 whereby the source of high energy particles can either be a nuclear reactor’s thermal neutron flux or it can be accelerated deuterons.

Na-24 has a physical half-life of 14.956 h, or approximately 15 hours after production. This relatively short half-life means that the majority of its radiation will be given off within 1-2 days of the patch being placed on the patient skin. By 7 days after production, more than 10 half-lives will have passed, meaning the activity will be reduced to 0.1% of the amount initially prescribed. Hence, there will be no appreciable radiation safety risks after 1-2 weeks from the initial patch production and placement upon a patient’s skin.

Sodium-24 emits beta-minus (electron) and gamma (photon) particles. An electron-Volt is a unit of energy commonly used in radiation physics and it is defined as the amount of energy given to an electron passing through a 1 Volt electrostatic potential. The beta particles have an average energy of about 500 kiloelectron Volts (“keV”) and a maximum energy of about 4 mega-electron Volts (“MeV”). The photons have primarily two energies of 1.4 MeV and 2.75 MeV, along with other energies that are clinically insignificant.

The beta particles from Sodium-24 are considered to have energy that is too low for therapeutic purposes, but the photons have a high energy that can be used for therapy to treat deeper cutaneous lesions of up to 1-2 cm below the surface. It is desirable to prevent the beta particles from hitting the patient as much as possible while allowing as many of the photons to pass through to the patient as possible. If the beta particles are allowed to impact the patient, the beta particles will increase the surface dose, considered the dose at 1 mm depth or shallower, by up to 20 times the dose at deeper depths, which is undesirable.

Gallium-66 (“Ga-66”) is a radionuclide which has been used experimentally in Positron Emission Tomography (PET) imaging. Ga-66 can be produced with a medical cyclotron that is commonly available in larger hospitals. Gallium 66 is a beta-plus (positron) emitter. The positrons (electrons with positive charge) have an average energy of about 1.8 MeV and a maximum of about 4 MeV. Ga-66 also emits many different gamma photons, with the most common photon being 1.04 MeV, but there is a wide distribution or variety of photons. Ga-66 has a shorter half-life of 9.5 hours when compared to Na-24. Ga-66 may hence be useful for treating very superficial lesions such as keloids. Without mixing with other isotopes, Y-90 may provide a deep enough electron beam penetration to provide an effective therapy for the treatment of shallow lesions such as keloids. In alternative embodiments, a mixture of Y-90 and Ga-66 may be utilized.

While Sodium-24 and Gallium-66 are presently deemed to be the most appropriate radioisotopes to be used in the topical radiation patch of the present invention, the patch is in principle capable of accepting any beta or gamma emitting radioisotope. Any collection of radioactive atoms may be sealed, in principle, into the interior of the sealed radiation patch. Hence, this invention is a novel type of sealed radiation source which may contain any natural or manmade beta particle or gamma photon emitting radioisotope.

In another embodiment, the present invention describes a source housing in FIGS. 5A and 5B which includes a radiation protection shield mechanism on the distal side and a radiation and depth-dose optimization bolus mechanism on the proximal side. In combination, this source housing also provides a watertight seal for the enclosed radiation source(s) that cannot be ruptured by a reasonable force as long as the patient follows reasonable instructions given by the clinical staff administering the patch.

FIG. 5A depicts a cross section of the radionuclide patch design which allows dosimetry optimization and a convenient dose delivery mechanism. The patch is designed to have an adhesive layer in contact with the skin and around the perimeter of a standard set of shapes such as circles, ellipsoids, or rectangles, or be custom shaped for a given body site such as across the cheeks and nose in one large area patch as depicted in FIG. 5B.

As depicted in FIGS. , 5A-5B, inside the patch is a radionuclide binding agent mechanism which contains the mixture of radionuclides chosen individually or in a specific mixture for a given lesion which is variable in thickness depending upon the desired dosimetry. It has a minimal protective film layer, or proximal side shielding layer, between the radionuclide binding agent mechanism and the skin which itself may be varied in dimension depending upon the energy of the decay particles coming from the radionuclides. The proximal side shielding layer can serve as a bolus material should the electron energies of a given isotope become high enough to require it for assuring an optimum prescription at a given depth, similar to bolus used routinely with high energy electron beams from linear accelerators. Bolus can also be used for tailoring the highest dose of a gamma ray emitter as well for consideration of different fractionation schemes, for example for bulkier lesions with deeper aspects.

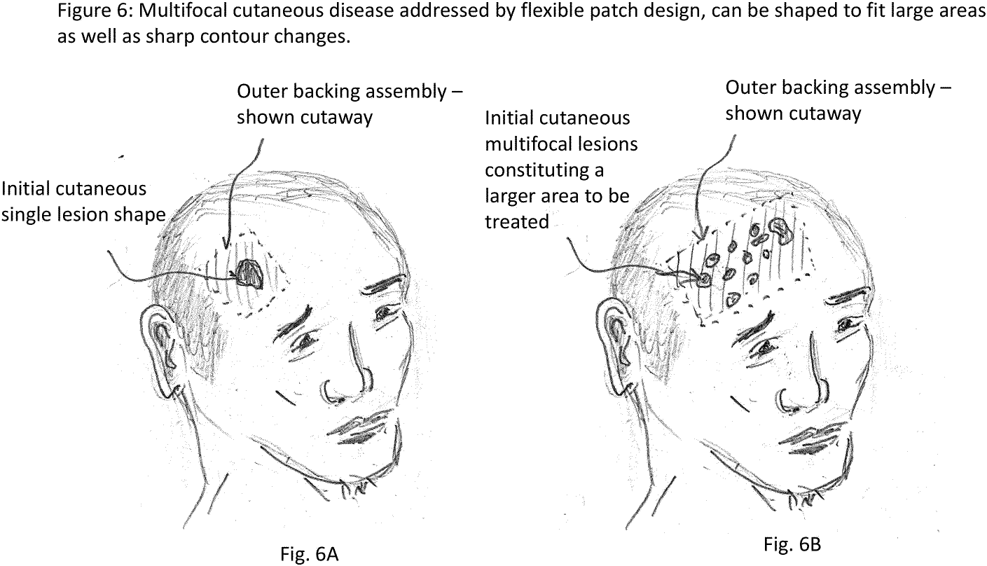

As depicted in FIGS. 6A-6B, in another embodiment of the present invention, the invention would accommodate a larger area of multifocal disease as is common in many countries with high patient volumes of skin cancers of the face, nose, head, and ears and is quite challenging to treat effectively with an acceptable cosmetic outcome. The flexible nature of the patch and the supporting materials show allow for it to bend and take the contour of the patient’s skin in challenging areas.

Table 1 was produced from equation 3 of the NRC publication NUREG 8.39 revision 1. The Gamma Constant of 1.93769 rem m^2 / (Ci-hr) for Na-24 was taken from the most conservative (highest)-valued source that could be found in the literature [17]. It is assumed per the NUREG report recommendations that 1 rem equals 10 mSv, and furthermore it is assumed that 1 Roentgen of exposure equals 1 rem of equivalent dose, which is a common assumption in gamma photon shielding calculations. The “Shielding Reduction Factor” refers to the percentage of the radiation fluence that is attenuated or reduced by the shielding that is built into the source housing. Hence, it may be seen that with a 90% reduction in radiation fluence exiting the distal side of the patch, an activity level of 100 mCi may be prescribed without exceeding the NRC release criteria. By adjusting the thickness and density of the shielding on the distal side of the patch, the allowable activity that can be prescribed may be increased or reduced accordingly while still being able to release the patient into the public following NRC release criteria. On the proximal side of the patch, a reasonable assumption can be made that the combination of the patient’s body tissues and the bolus on the proximal side of the patch combine together to provide above and beyond the shielding requirements that are necessary to release the patient into the public, since the patient’s body tissues will be on the order of 10-20 cm in thickness on average in addition to the thickness of the source housing or bolus.

In another embodiment of the present invention, the BED, dwell time, and fractionation scheme for the low dose rate brachytherapy created by the radiation patch may be optimized to match or exceed other fractionation schemes in widespread clinical use for other treatment modalities. The Relative Effectiveness per unit Dose (R.E.) of the radiation patch may be calculated using equation (d) of reference [13]. The BED is the R.E. (defined in [13]) times the total dose for a given fractionation scheme. In other words, the BED = R.E.^{∗}nd, where n is the number of patch treatments in the fractionation scheme and d is the physical absorbed dose per fraction. ASTRO is the American Society for Radiation Oncology, and this organization published a report of guidelines for fractionation of skin cancer. The ASTRO skin fractionation table was published in reference [5], Table 6. It is possible to compute the equivalent dose fractionation schemes for the Na-24 skin patch that produce the same BED as the BED for the fractionation schemes in the ASTRO table using the equations in [13]. Table 2 herein shows the equivalent fractionation schemes for the Na-24 patch with 6 mm bolus for a prescribed depth of 3 mm. The activity of Na-24 required to deliver the BED is shown on the right of the Table. This activity was calculated using the equations of [13]. The alpha/beta ratio of the tumor is assumed to be 10 Gy and the “mu” value or tissue repair constant for skin is assumed to be 0.32 h⁻¹.

In one Embodiment, the apparatus of the present invention provides a ready means to deliver / expose the patient to particles and energies of radionuclide isotopes via an adhesive or applied patch with radionuclide therein. Another embodiment describes the use of radiological “bolus” material to optimize the flux or fluence of radiation particles incident on the patient for optimal radiobiological and therapeutic outcomes. Another embodiment presents a methodology for the selection of the radionuclide isotope or combinations of radionuclide isotopes for use within the apparatus for patient treatment of a given cutaneous lesion. In yet another embodiment, the application of a “shield” material on the external or distal side of the wearable patch is described; such shield material having the dual purpose of providing radiation protection to “members of the public” as per the requirements of 10 C.F.R. 35 of the United States Code and furthermore to qualify the enclosed radioactive material and its chemical substrate as a “sealed source” per the same 10 C.F.R. 35 regulations. Another embodiment of the invention claims the use of the radioisotopes Sodium-24 and Gallium-66, being the isotopes of Sodium (“Na”) with atomic number 11 and mass number 24 and Gallium (“Ga”) with atomic number 31 and mass number 66, and any combination of said isotopes, as the radioactive atoms comprising the source of particles emanating from a wearable superficial medical or veterinary patch.

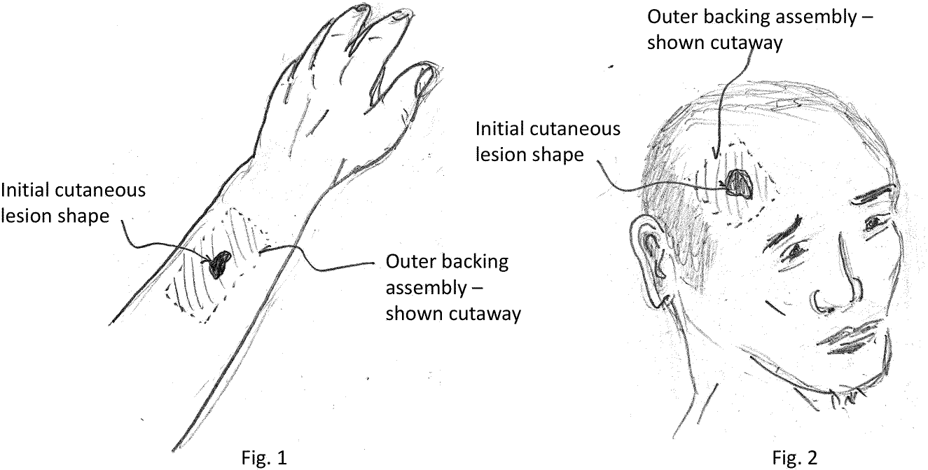

In one embodiment, this invention relates to the application of a ready supply of a wide variety of radionuclides specifically for the treatment of cutaneous lesions. Cutaneous lesions exist within the first centimeter of tissue from the body surface. FIGS. 1, 2 depicts some common sizes, shapes, and locations for lesions though they can appear anywhere on the skin surface, they are mostly apparent on the extremities and exposed portions of face, head and neck. As depicted in FIG. 1, a cutaneous lesion is shown on a human forearm. As depicted in FIG. 2, a cutaneous lesion is shown on a human forehead. The lesions can be benign or cancerous processes and radiation has long been established as an effective curative treatment including both high energy electrons and photons of various energies (note - for example MV tangent pair or tomotherapy). This invention seeks to replicate the excellent curative and cosmetic benefits established with ionizing radiation sources from linear accelerators and X-ray tubes but achieved with the use of selective radionuclides alone or in combination.

In another embodiment, proper use of a material and technique known in radiotherapy as “bolus” involves the superficial placement of a material with a known thickness and mass density which effectively shifts the percent depth-dose curves in the patient’s tissues towards the surface, as the radiation impinges upon the bolus material first and the beam begins to deposit dose in the bolus before it encounters the actual skin surface. The fact that various materials may be used as bolus stems from the concept of “radiological depth” or “density thickness,” which is defined as the thickness of a given material divided by its mass density. This radiological depth has units of mass per area (e.g. grams per cm squared) and is a more significant factor that determines the attenuation of radiation in a given material than is the physical thickness or the mass density alone. A bolus may be affected by using a certain thickness of a higher atomic number material like lead or aluminum which gives the same apparent radiological depth from the standpoint of the impact on the radiation beam or radiation from the treatment wafer after transiting the bolus material as compared to a thicker portion of tissue equivalent material which would make the patch thicker and bulkier. For example, usage of a bolus with Pd-103 can affect a shift of the depth dose curve back to any depth desired for a given tumor thickness by applying a bolus material of that thickness to the patient at the beam entry or patch application point. It is also possible to mix two different isotopes in with some abundance ratio which would yield a combined percent depth-dose curve based on the relative abundance of in this example, I-125 and Pd-103, in addition to the effect of the bolus on the resulting percent depth-dose curve. The combined depth dose curve is some optimal curve to place dose closer to the surface or farther from the surface or more uniform throughout from surface to prescription depth depending upon the clinician’s intent.

While the use of bolus is ubiquitous in “external beam” radiotherapy, it has not yet been used as a way to optimize the fluence of radiation particles coming from an adjacent, superficially applied radioisotope source. One embodiment of the present invention is the use of a bolus material between the radioactive substrate and the patient’s skin which is designed to prevent undesirable radiation particles from imparting energy into the patient while allowing other radiation particles with more desirable properties to pass through the bolus and to deposit dose in the target. One exemplary embodiment of an undesirable particle type which the bolus will filter out of the beam is a low-energy electron with energy of approximately a few hundred kilo-electronVolts (keV). An exemplary embodiment of a desirable particle that may pass through the bolus material with only a small amount of attenuation in its fluence is a high energy photon with energies in the range of 0.1 to 3 mega-electron Volts (MeV). This differential attenuation of the undesirable electron (or beta) particle fluence without causing significant attenuation of the desirable photon (or gamma) particle fluence is the purpose of the bolus material on the proximal side of the wearable patch, in addition to the mechanical purpose of helping to seal the radioactive substrate into the interior of the patch. The particles which may be differentially attenuated by the bolus material on the proximal side of the patch are not limited to the aforementioned particle types, which are exemplary embodiments of how the bolus effect may be used to optimize the radiation fluence incident upon the patient from a wearable radioisotope patch.

FIG. 8 presents the PDD curves for Na-24 for both the gamma (photon) particles and the total unshielded PDD for the beta-minus (electron) and gamma (photon) particles. Curves for 0.3 cm H20 shielded and 0.6 H20 shielded are also presented. As a comparison, 0.3 cm of H20 is radiologically equivalent (in terms of density thickness) to 0.026 cm of Lead (Pb), and 0.6 cm of H2O is radiologically equivalent to .053 mm of Lead (Pb). This can be calculated by dividing the physical thickness in centimeters by the density of Pb which is 11.34 grams per cubic centimeter. As can be seen by one skilled in the art, the “Total” Na-24 PDD becomes closer and closer to the “unshielded gamma” Na-24 PDD as more shielding is added. This means that the electrons (betas-minus) are contributing less and less to the PDD as more shielding material is placed between the radionuclide source and the skin surface. Stated another way, FIG. 8 presents the separate beta-minus (electron) and gamma (photon) PDDs because the beta-minus can be filtered or shielded out and the patient may then be treated with only the gammas to achieve a deeper PDD for a given amount of material. Of keynote in FIG. 8, the Na-24 Gamma (photons) only (beta-minus electrons shielded out) almost exactly overlays 50 kVp setting of the most commonly used skin superficial radiotherapy machine currently in place in hundreds of clinics. Commercially speaking, 50 kVp delivers the most commonly used dose curve for all cutaneous non-melanoma skin cancers which are of a limited extent within the epidermis and stopping somewhere before the end of the dermis. Currently in the industry, more machines are in place treating more lesions of this type than any other technology.

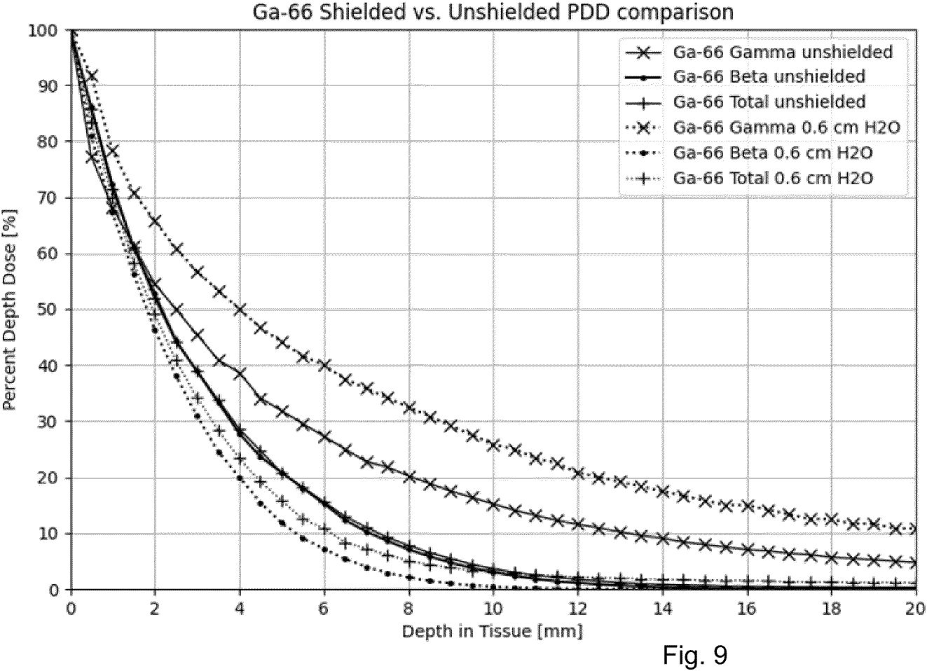

FIG. 9 presents the PDD curves for Ga-66 for both the gamma (photon) particles and the total unshielded PDD for the beta-plus (positron) and gamma (photon) particles. It can be inferred from the FIG. 9 that the dose deposited from Ga-66 is dominated by the positrons and not the gamma photons. Hence, Ga-66 is ideal for shallow lesions such as keloids.

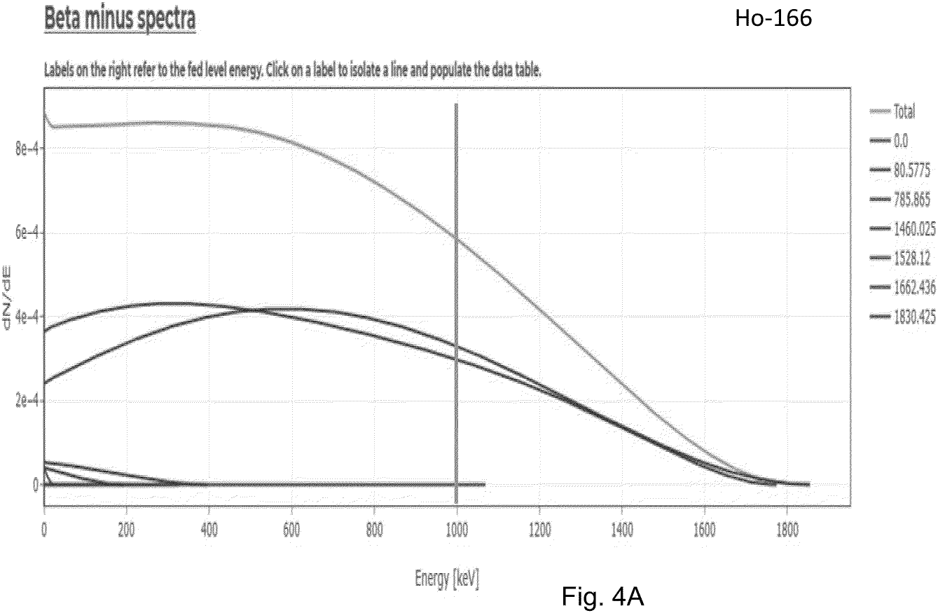

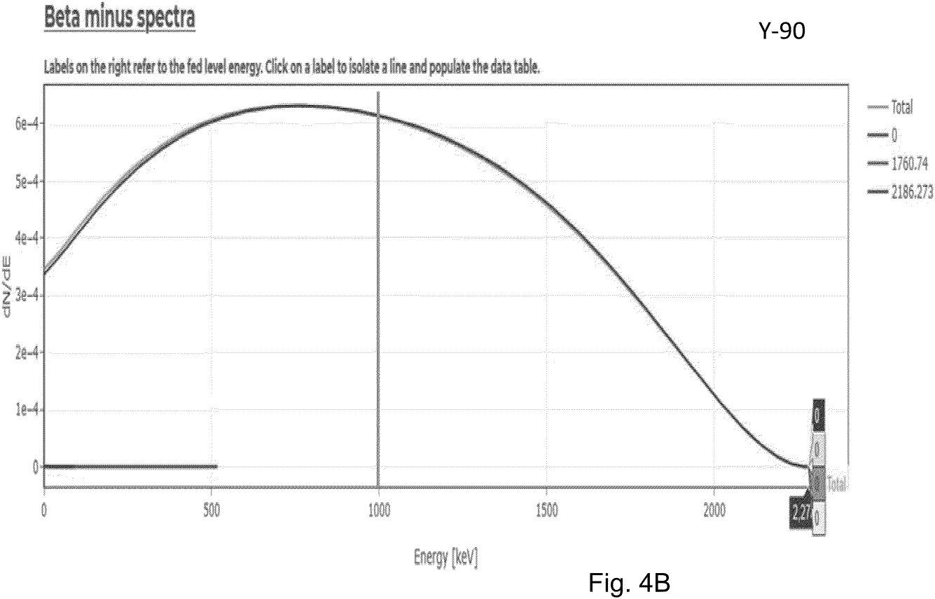

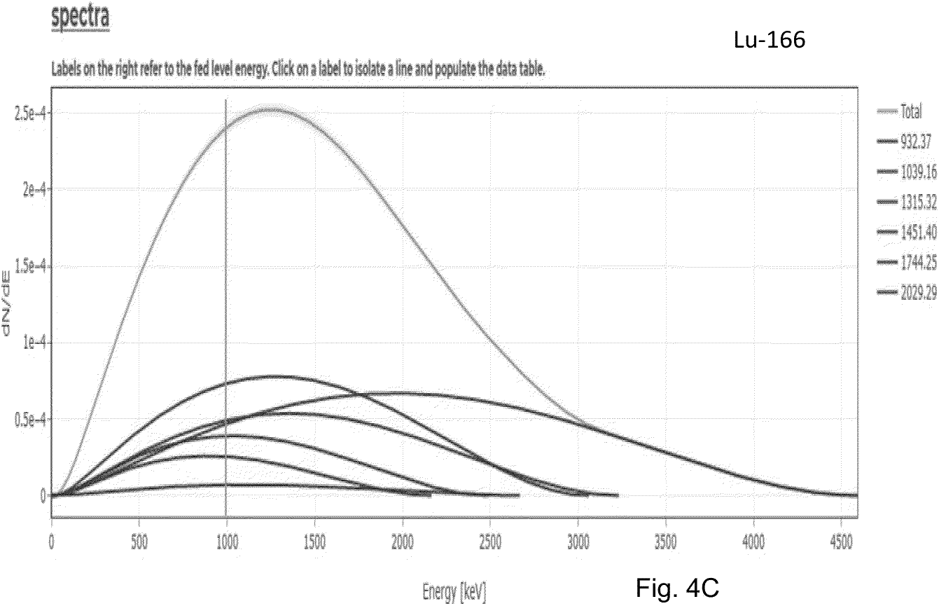

In another embodiment of the present invention, in the future some as yet un-isolated isotopes may be able to be created in the modern accelerators or reactors which having known decay schemes further enhancing and optimizing the ability to build a wearable patch and give patient specific optimal dose delivery. One example is Lu-166 which decays via a very high energy electron emission at 4.5 MeV approximately. As depicted in FIGS. 4A, 4B, and 4C, if we compare Ho-166 to Y-90 and Lu-166 in terms of decay spectra of electrons one can see how an isotope such as Lu-166, as it is commercially available, could enable continued evolution of this approach of optimizing and mixing isotopes. FIG. 4A shows the decay spectra from Ho-166. FIG. 4B shows the decay spectra from Y-90. And FIG. 4C shows the decay spectra from Lu-166. Each FIGS. 4A, 4B, and 4C show a reference line at 1 MeV energy to show the relative differences of the electrons emitted from each. Were Lu-166 available to measure the percent depth dose one could reasonably expect it to penetrate deeper and perhaps higher even than 6 MeV electrons depth dose. This can also be a convenient approach to have one isotope with excess depth dose range and then utilize the disclosed bolus thickness to customize it to a given patient’s lesion depth.

In another embodiment of the present invention, Sodium-24 (“Na-24”) and/or Gallium-66 (“Ga-66”) may be used as the radioactive isotope (“radionuclide”), either singularly or in combination with respective fractional percentages from 0% to 100%. Both Na-24 and Ga-66 have been used in nuclear medicine for imaging. However, Na-24 and/or Ga-66 have not been combined or singularly used for a therapeutic application for cutaneous lesions. The use of radionuclides eliminates the need for “external beam” radiotherapy using expensive X-ray machines or linear accelerators for superficial skin lesions. It also allows for a radiation dosing approach that is commonly referred to as low dose-rate radiotherapy (“LDR”) or LDR Brachytherapy whereby a given radioactive source is applied to lesions in the body over some part or all of the isotope’s decay timescale and the source(s) are placed adjacent to the lesion in a fixed relationship for the entire radiation period. Generally these sources to be classified as LDR sources, they must deliver a clinical dose rate of less than 0.5 Gray per hour. [18]

In the present invention, experience in radiation oncology and physics informs one skilled in the art that available medical isotopes placed within a patch type device can create a dosimetrically suitable alternative to using a linear accelerator or X-ray tube-based skin cancer treating device. The radionuclide patch of the present invention has the potential to provide an equivalent or superior medical treatment to a 50 kVp X-ray beam without the need for an X-ray machine. A radionuclide patch would allow any trained physician (depending on what the applicable country, the U.S. NRC, and U.S. State consider adequate training) to prescribe radiation to skin cancer without needing to purchase expensive X-ray tubes and service contracts. The physician’s practice would only need to order patch(es) individually for each patient’s intended course of treatment, which may entail one or more patches placed serially in time as per the prescription to deliver the desired BED for the isotope being utilized. The radionuclide patch may be supplied with software to calculate the activity needed to deliver the prescribed dose that the physician desires. This would completely supplant a 50 kVp X-ray treatment so commonly used today. Future developments of suitable candidate isotopes such as Na-24, Ga-66, and Lu-166 could also be utilized in the disclosed cutaneous lesion radionuclide patch singularly or in combination.

While there has been shown a preferred embodiment of the present invention, it is to be understood that certain changes may be made in the forms and arrangement of the elements of radionuclide isotopes available for the treatment of cutaneous lesions of the human body without departing from the underlying spirit, scope, and essential characteristics of the invention. The present embodiment is therefore, to be considered as merely illustrative and not restrictive, the scope of the invention being indicated by the claims rather than the foregoing description, and all changes which come within the meaning and range of equivalence of the claims are therefore intended to be embraced therein.

What is claimed is:

1. A radioactive dermatological patch, the patch designed to topically treat cutaneous skin lesions in patient tissue, the patch comprising;
a layer of a radionuclide with a nonreactive binding agent to form a treatment wafer, the treatment wafer placed within the radioactive dermatological patch;
a high Z distal shielding layer placed adjacent the side of the treatment wafer to be away from the patient tissue, the distal shielding layer attenuating the energy of the radionuclide from an environment external to the patient; and
a high Z proximal patient shielding layer placed adjacent the side of the treatment wafer to be adjacent the patient tissue.

2. The radioactive dermatological patch of claim 1, wherein an additional window shielding layer of high Z shielding is placed between the treatment wafer and the proximal patient shielding layer, the window layer of high Z shielding comprising a cutout opening within the window shielding layer in the shape of the cancerous tissue to be treated by the radionuclide and the window layer comprising a substantially solid layer of high Z shielding above the remaining healthy patient tissue.

3. The radioactive dermatological patch of claim 1, wherein an additional window shielding layer of high Z shielding is placed between the distal shielding layer and the proximal patient shielding layer, the window shielding layer comprising a cutout opening within the window shielding layer in the shape of the cancerous tissue to be treated by the radionuclide and the window shielding layer comprising a substantially solid layer of high Z shielding above the remaining healthy patient tissue; and werein the treatment wafer is applied within the cutout of the window shielding layer.

4. The radioactive dermatological patch of claim 1, wherein the radionuclide comprises at least one of: Na-24, Ga-66, or any combination thereof.

5. The radioactive dermatological patch of claim 1, wherein the nonreactive binding agent is comprised of a silicone rubber.

6. The radioactive dermatological patch of claim 2, wherein the window shielding layers of high Z shielding is comprised of at least one of: lead, tungsten, iron, silver, gold, platinum, copper, brass, or any combination thereof.

7. The radioactive dermatological patch of claim 3, wherein the window shielding layers of high Z shielding is comprised of at least one of: lead, tungsten, iron, silver, gold, platinum, copper, brass, or any combination thereof.

8. The radioactive dermatological patch of claim 1, wherein the radionuclide and nonreactive binding agent are formed in a substantially circular treatment wafer within a mold.

9. The radioactive dermatological patch of claim 1, wherein the radionuclide and nonreactive binding agent are formed in a substantially elliptical treatment wafer within a mold.

10. The radioactive dermatological patch of claim 1, wherein the radionuclide and nonreactive binding agent are formed in a custom shape corresponding to the shape of the cutaneous skin lesions in the patient tissue.

11. The radioactive dermatological patch of claim 1, wherein the treatment wafer shape corresponds to a portion of the upper cranium of the patient.

12. The radioactive dermatological patch of claim 1, wherein the distal shielding layer is comprised of at least one of: lead, tungsten, iron, silver, gold, platinum, copper, brass, or any combination thereof.

13. The radioactive dermatological patch of claim 1, wherein the proximal shielding layer is comprised of at least one of: lead, tungsten, iron, silver, gold, platinum, copper, brass, or any combination thereof.

14. The radioactive dermatological patch of claim 2, wherein an adhesive binds at least one of; the proximal shielding layer to the window shielding layer, the distal shielding layer to the window shielding layer, the proximal shielding layer to the treatment wafer, the distal shielding layer to the treatment wafer, the proximal shielding layer to the distal shielding layer.

15. The radioactive dermatological patch of claim 3, wherein an adhesive binds at least one of; the proximal shielding layer to the window shielding layer, the distal shielding layer to the window shielding layer, the proximal shielding layer to the distal shielding layer.

16. The radioactive dermatological patch of claim 1, wherein the proximal shielding layer substantially attenuates the electron energy emitted from the radionuclide.
